(19) United States
(12) Patent Application Publication
(10) Pub. No.: US 2024/0382539 A1
(43) Pub. Date: Nov. 21, 2024

(54) USE OF INTESTINAL PROBIOTIC IN PREPARATION OF MEDICAMENT THAT PROMOTES METABOLISM OF ELLAGIC ACID INTO UROLITHIN A, PHARMACEUTICAL COMPOSITION AND USE THEREOF

(71) Applicant: Zhejiang University, Hangzhou (CN)

(72) Inventors: Haibo PAN, Hangzhou (CN); Laiming ZHANG, Hangzhou (CN); Shiguo CHEN, Hangzhou (CN); Xingqian YE, Hangzhou (CN); Donghong LIU, Hangzhou (CN); Tian DING, Hangzhou (CN)

(73) Assignee: Zhejiang University, Hangzhou (CN)

(21) Appl. No.: 18/229,592

(22) Filed: Aug. 2, 2023

(30) Foreign Application Priority Data
May 19, 2023 (CN) 2023105790477

(51) Int. Cl. A61K 35/747; A61K 31/366; A61K 35/741; A61K 35/744; A61K 35/745; A61P 1/14

(52) U.S. Cl. CPC A61K 35/747; A61K 31/366; A61K 35/741; A61K 35/744; A61K 35/745; A61P 1/14

(57) ABSTRACT

The present disclosure provides use of an intestinal probiotic in preparation of a medicament that promotes metabolism of ellagic acid (EA) into urolithin A, a pharmaceutical composition and use thereof, and belongs to the technical field of biomedicine. In the present disclosure, probiotics capable of changing metabotypes of EA are selected from a plurality of probiotics. These probiotics alone or in combination can change an intestinal flora structure of a metabotype B and/or 0 population, thereby improving the efficiency of the metabolism of EA into urolithin A and increasing a yield of urolithin A. The present disclosure provides an intestinal probiotic for involving in and promoting the metabolism of EA into urolithin A, and provides a new idea for the treatment of diabetes, obesity, senescence, and diseases caused by central nervous system lesions and viral infection using urolithin A as an active pharmaceutical ingredient.

### CROSS REFERENCE TO RELATED APPLICATION

This patent application claims the benefit and priority of Chinese Patent Application No. 2023105790477 filed with the China National Intellectual Property Administration on May 19, 2023, the disclosure of which is incorporated by reference herein in its entirety as part of the present application.

### TECHNICAL FIELD

The present disclosure belongs to the technical field of biomedicine, and particularly relates to use of an intestinal probiotic in preparation of a medicament that promotes metabolism of ellagic acid (EA) into urolithin A, a pharmaceutical composition and use thereof.

### BACKGROUND

Ellagic acid (EA) is a widely sourced and easily extracted natural polyphenol present as ellagitannin (ET) in berries (pomegranate, strawberry, etc.) and nuts (walnut, chestnut, etc.). In the gastrointestinal tract, ET is hydrolyzed to release hexahydroxydiphenyl acid and rapidly condensed into ellagic acid. EA is a strong polar molecule that is hardly absorbed and used by the gastrointestine. Therefore, EA cannot exert effective bioactivity due to its low bioavailability. Specific microflora in the human intestinal tract can effectively metabolize EA and produce a range of urolithins, which can be effectively used and produce a range of probiotic activity compared with EA. For example, Japanese patent JP2022190124 indicates efficacy of urolithin A in diabetes. obesity, senescence, and central nervous system lesions. Meanwhile, Chinese patent CN111939152A discloses use of EA metabolite urolithin A in preparation of an anti-enteroviral medicament.

Current research has shown that EA can be metabolized into different urolithin derivatives in the colon. Herein, urolithin A, isourolithin A and urolithin B are metabolic end products of EA. The metabotype of EA in population can be divided into metabotypes A, B, and 0 according to the differences in metabolic end products of EA in different populations; metabotype A population finally produce urolithin A. metabotype B population produce urolithin A, urolithin B and isourolithin A, and metabotype 0 population cannot metabolize EA to produce urolithins (Tomás-Barberán F A, Garcia-Villalba R, Gonzalez-Sarrias A, et al. Ellagic acid metabolism by human gut microbiota: consistent observation of three urolithin phenotypes in intervention trials, independent of food source, age, and health status [J]. Journal of Agricultural and Food Chemistry, 2014, 62(28): 6535-6538.). Therefore, in metabotype 0 population, EA cannot be metabolized into urolithin A that produces effective probiotic activity; in metabotype B population, the yield of urolithin A decreases and cannot reach an effective dose. This leads to limited use of EA in metabotypes 0 and B populations. The difference in metabolic end product among populations is related to the difference in human gut microflora composition. Therefore, it is urgent to develop a method for improving intestinal flora structures of metabotypes 0) and B populations to promote the metabolic transformation of EA.

Intestinal probiotics can effectively improve the bioactivity of EA: Chinese patent CN115299611 discloses a composition of Bacillus coagulans combined with EA and use thereof. EA and Bacillus coagulans are directly mixed to form a composition that has excellent efficacy in relieving hypercholesteremia and preventing obesity. US20210038654 discloses a composition for modulating gut microflora populations, enhancing drug potency and treating cancer, and methods for making and using same. Bacteria from 16 genera including Faecalibacterium, Brucella, Bifidobacterium, Bacteroides, Ruminococcus, E., Gordonia and the like are compounded with EA to form a composition. This composition can effectively increase the antitumor activity of EA, but does not relate to the effect of the composition on population metabotypes. WO2019212997 discloses biosynthetic preparation of urolithin compounds and use thereof. EA, whole or skim milk, a yoghurt starter, and probiotics including Lactobacillus acidophilus, Bifidobacterium lactis, Lactobacillus plantarum, Lactobacillus salivarius and the like are mixed and fermented in a jar fermentor at 21° C. for 14 h to increase the biotransformation rate of EA. However, the fermentation system neither mimics the human gut system nor relates to the improvement of population metabotypes to increase the yield of urolithin A, and the transformation rate of the metabolism of EA into urolithin A by this system is lower than 0.1%. Therefore, there is no research on the compounding of intestinal flora with EA to improve population metabotypes to promote EA metabolism and effectively increase the production of urolithin A.

### SUMMARY

In view of this, the present disclosure aims to provide use of an intestinal probiotic in promotion of metabolism of EA into urolithin A. The metabolism of EA in guts of metabotypes 0) and B populations into urolithin A is promoted by optimizing and selecting a composition of the intestinal probiotic.

The present disclosure aims to provide a pharmaceutical composition. Compounding intestinal probiotics capable of promoting metabolism of EA into urolithin A with EA may change EA metabotypes in metabotypes 0 and B populations to increase the metabolic content of urolithin A. Thus, the pharmaceutical composition can be used in the treatment of diabetes, obesity, senescence, central nervous system lesion and the like.

The present disclosure provides an intestinal probiotic, including at least one probiotic selected from the group consisting of Lactobacillus acidophilus (L. acidophilus), Lactobacillus plantarum (L. plantarum), Lactobacillus rhamnosus (L. rhamnosus), Bifidobacterium breve (B. breve), Bifidobacterium bifidum (B. bifidum), Bifidobacterium longum (B. longum), Bacteroides fragilis (B. fragilis), Bacteroides vulgatus (B. vulgatus), Megasphaere elsdenii (M. elsdenii), Ruminococcus gnavus (R. gnavus), Weissella confusa (W. confusa), Enterococcus faecalis (E. faecalis), and Akkermansia muciniphila (A. muciniphila).

In some embodiments, the intestinal probiotic includes the following combinations: a first intestinal probiotic formed by L. acidophilus, L. plantarum and L. rhamnosus, a second intestinal probiotic formed by B. breve, B. bifidum and B. longum, a third intestinal probiotic formed by B. fragilis and B. vulgatus, a fourth intestinal probiotic formed by M. elsdenii, R. gnavus and W. confusa, a fifth intestinal probiotic formed by E. faecalis and A. muciniphila, a sixth intestinal probiotic formed by B. breve, B. fragilis and A. muciniphila, and a seventh intestinal probiotic formed by B. longum, R. gnavus and B. vulgatus.

In some embodiments, a ratio of viable counts of microbial species within the first intestinal probiotic, the second intestinal probiotic, the six intestinal probiotic, and the seventh intestinal probiotic or the fourth intestinal probiotic is (1-3):(1-3):(1-3); and
a ratio of viable counts of microbial species within the third intestinal probiotic and the fifth intestinal probiotic is (1-3):(1-3).

The present disclosure provides a pharmaceutical composition for increasing urolithin A content, including the intestinal probiotic and EA.

In some embodiments, the intestinal probiotic and EA have a volume-mass ratio of 1 mL:(5-20) g; and
the intestinal probiotic has a viable cell concentration of 1×10⁸ to 5×10⁸ colony-forming unit (CFU)/mL.

In some embodiments, EA has a structure of formula I:

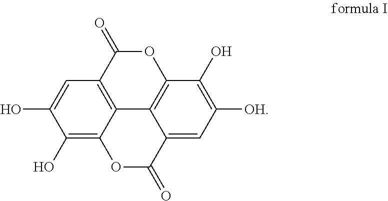 [SMILES: O=c1oc2c(O)c(O)cc3c(=O)oc4c(O)c(O)cc1c4c23]

The present disclosure provides use of the intestinal probiotic or the pharmaceutical composition in preparation of a medicament that promotes metabolism of EA into urolithin A.

In some embodiments, a population metabotype that promotes metabolism of EA into urolithin A is metabotype 0 and/or B.

Furether provided are use of at least one selected from the group consisting of the first intestinal probiotic to the seventh in preparation of a medicament that promotes metabolism of EA into urolithin A in a metabotype B population; and
use of at least one selected from the group consisting of the second intestinal probiotic, the third intestinal probiotic, the fifth intestinal probiotic and the seventh intestinal probiotic in preparation of a medicament that promotes metabolism of EA into urolithin A in a metabotype 0 population.

The present disclosure provides use of the intestinal probiotic or the pharmaceutical composition in preparation of a medicament for prophylaxis and/or treatment of at least one disease selected from the group consisting of diabetes, obesity, senescence, and diseases caused by central nervous system lesions and viral infection.

In some embodiments, patients with the diseases belong to a metabotype B population and/or a metabotype 0 population of EA metabolism.

The present disclosure provides an intestinal probiotic, including at least two probiotics selected from the group consisting of L. acidophilus, L. plantarum, L. rhamnosus, B. breve, B. bifidum, B. longum, B. fragilis, B. vulgatus, M. elsdenii, R. gnavus, W. confusa, E. faecalis, and A. muciniphila. In the present disclosure, probiotics capable of changing metabotypes of EA are selected from a plurality of probiotics. These probiotics alone or in combination can change an intestinal flora structure of a metabotype B and/or 0 population, thereby improving the efficiency of the metabolism of EA into urolithin A and increasing the yield of urolithin A. Visibly, the present disclosure provides an intestinal probiotic that involves in and promotes the metabolism of EA into urolithin A, and provides a new idea for the improvement of metabotypes of EA and the treatment of diabetes, obesity, senescence, and diseases caused by central nervous system lesions and viral infection using urolithin A as an active pharmaceutical ingredient.

### BRIEF DESCRIPTION OF THE DRAWINGS

FIG. 1 illustrates screening results of metabolic probiotics in a metabotype B population;

FIG. 2 illustrates screening results of metabolic probiotics in a metabotype 0 population;

FIG. 3 illustrates changes of urolithin A content through promotion of EA metabolism in feces of metabotype B population by different combinations of probiotics in Examples 2 to 8; note: ****P<0.0001 versus control group;

FIG. 4 illustrates changes of urolithin A content through promotion of EA metabolism in feces of metabotype 0 population by different combinations of probiotics in Examples 2 to 8; note: no urolithin A is produced in metabotype 0 microfloras of control group and Examples 1 and 4; and

FIG. 5 illustrates changes urolithin A content through promotion of EA metabolism in animal experiments by different combinations of probiotics in Examples 2 to 8.

### DETAILED DESCRIPTION OF THE EMBODIMENTS

The present disclosure provides an intestinal probiotic including at least one probiotic selected from the group consisting of L. acidophilus, L. plantarum, L. rhamnosus, B. breve, B. bifidum, B. longum, B. fragilis, B. vulgatus, M. elsdenii. R. gnavus. W. confusa, E. faecalis, and A. muciniphila.

In the present disclosure, 13 intestinal probiotics capable of changing a metabotype of EA and increasing urolithin A content are optimized and selected from at least 20 known intestinal probiotics.

In the present disclosure, the intestinal probiotic preferably includes at least one of the following combinations: a first intestinal probiotic formed by L. acidophilus, L. plantarum and L. rhamnosus, a second intestinal probiotic formed by B breve, B. bifidum and B. longum, a third intestinal probiotic formed by B. fragilis and B. vulgatus, a fourth intestinal probiotic formed by M. elsdenii, R. gnavus and W. confusa, a fifth intestinal probiotic formed by E. faecalis and A. muciniphila, a sixth intestinal probiotic formed by B. breve, B. fragilis and A muciniphila, and a seventh intestinal probiotic formed by B. longum, R. gnavus and B. vulgatus; more preferably the second intestinal probiotic, the third intestinal probiotic, the fifth intestinal probiotic, and the seventh intestinal probiotic; further preferably the seventh intestinal probiotic and the third intestinal probiotic; and most preferably the seventh intestinal probiotic. A ratio of viable counts of microbial species within the first intestinal probiotic, the second intestinal probiotic, the six intestinal probiotic, the seventh intestinal probiotic or the fourth intestinal probiotic is preferably (1-3):(1-3):(1-3), and more preferably 1:1:1; and a ratio of viable counts of microbial species within the third intestinal probiotic or the fifth intestinal probiotic is (1-3):(1-3), and more preferably 1:1. In an embodiment of the present disclosure, type strains, i.e., L. acidophilus ATCC 4356; L. plantarum ATCC 8014; L. rhamnosus ATCC 53103; B. breve ATCC 15700; B. bifidum ATCC 29521; B. longum ATCC 15707; B. fragilis ATCC 25285; B. vulgatus ATCC 8482; M. elsdenii ATCC 25940; R. gnavus ATCC 29149; W. confusa ATCC 10881; E. faecalis ATCC 29212; and A. muciniphila ATCC BAA-835, are used as representatives of microbial species.

The present disclosure provides a pharmaceutical composition for increasing urolithin A content, including an intestinal probiotic and EA. The intestinal probiotic is selected from the group consisting of the intestinal probiotics used in the foregoing technical solution.

In the present disclosure, the intestinal probiotic and EA have a volume-mass ratio of 1 mL:(5-20) g, more preferably 1 mL:(8-18) g, and further preferably 1 mL:12 g. The intestinal probiotic has a viable cell concentration of 1×10⁸ to 5×10⁸ CFU/mL, more preferably 2×10⁸ to 4×10⁸ CFU/mL, and most preferably 3×10⁸ CFU/mL.

In the present disclosure, the intestinal probiotic is preferably obtained by activation. A culture medium for the activation of the intestinal probiotic is preferably Brain Heart Infusion (BHI) Broth. There is no particular limitation on the activation in the present disclosure, and an activation method known in the art may be used.

In the present disclosure, EA preferably has a structura of formula I:

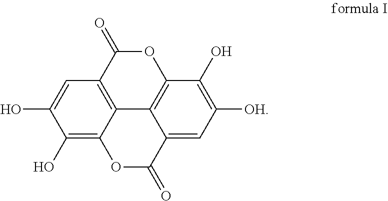 [SMILES: O=c1oc2c(O)c(O)cc3c(=O)oc4c(O)c(O)cc1c4c23]

EA may be extracted from one or more selected from the group consisting of pomegranate, blackberry, strawberry, pomegranate, wolfberry, raspberry, white oak acorn, cranberry, and pecan. In an embodiment of the present disclosure, EA is purchased from Sigma-Aldrich, Inc.

The present disclosure provides use of the intestinal probiotic or the pharmaceutical composition in preparation of a medicament that promotes metabolism of EA into urolithin A.

In the present disclosure, a population metabotype that promotes the metabolism of EA into urolithin A is preferably metabotype 0 and/or B. Both in vivo and in vitro experiments in the present disclosure indicate that compounding of the intestinal probiotic with EA can change a metabotype of EA in intestinal metabolic microflora, while a metabotype that never produces or produces low-yield urolithin A (metabotype 0 or B) is transformed into metabotype A. This provides a possibility for those who do not metabolize to produce urolithin A to receive disease treatment with metabolite urolithin A.

The present disclosure provides use of at least one selected from the group consisting of the first intestinal probiotic to the seventh intestinal probiotic preferably in preparation of a medicament that promotes the metabolism of EA into urolithin A in a metabotype B population; and at least one selected from the group consisting of the second intestinal probiotic, the third intestinal probiotic, the fifth intestinal probiotic and the seventh intestinal probiotic in preparation of a medicament that promotes the metabolism of EA into urolithin A in a metabotype 0 population.

Urolithin A has therapeutic effects on a plurality of diseases (Toney. A. M., R. Fan, Y. B. Xian, V. Chaidez, A. E. Ramer-Tait, and S. Chung. 2019. Urolithin A, a gut metabolite, improves insulin sensitivity through augmentation of mitochondrial function and biogenesis. Obesity (Silver Spring, MD) 27(4):612-620; Ryu, D., L. Mouchiroud, P. A. Andreux, E. Katsyuba, N. Moullan, A. A. Nicolet-Dit-Félix, E. G. Williams, P. Jha, G. Lo Sasso, D. Huzard, et al. 2016. Urolithin A induces mitophagy and prolongs lifespan in C. elegans and increases muscle function in rodents. Nature Medicine 22(8):879-888; Gong. Z., J. Y. Huang, B. Xu, Z. R. Ou, L. Zhang, X. H. Lin. X. J. Y e, X. J. Kong, D. H. Long, X. D. Sun, et al. 2019. Urolithin A attenuates memory impairment and neuroinflammation in APP/PSI mice. Journal of Neuroinflammation 16(1):13.), and the pharmaceutical composition can increase urolithin A content in the body. Therefore, the present disclosure provides use of the intestinal probiotic or the pharmaceutical composition in preparation of a medicament for prophylaxis and/or treatment of at least one disease selected from the group consisting of diabetes, obesity, senescence, and diseases caused by central nervous system lesions and viral infection.

In the present disclosure, patients with the diseases preferably belong to a metabotype B population and/or a metabotype 0 population of EA metabolism. In the present disclosure, the metabotype 0 population and/or the metabotype B population cannot metabolize to produce or can metabolize low-content urolithin A. In view of this problem, intestinal probiotics in the pharmaceutical composition can improve microbial community structure and thus metabotypes of EA, thereby increasing the yield of urolithin A.

The use of an intestinal probiotic in preparation of a medicament that promotes metabolism of EA into urolithin A, the pharmaceutical composition and the use thereof will be described in detail below with reference to examples, but they should not be construed as limiting the claimed scope of the present disclosure.

### Descriptions of Sources of Microbial Species and Drugs

L. acidophilus ATCC 4356 strain, L. plantarum ATCC 8014 strain, L. rhamnosus ATCC 53103 strain, L. salivarius ATCC 11742 strain, B. breve ATCC 15700 strain, B. bifidum ATCC 29521 strain, B. longum ATCC 15707 strain, B. fragilis ATCC 25285 strain, B. vulgatus ATCC 8482 strain, B. caccae ATCC 43185 strain, B. thetaiotaomicron ATCC 29741 strain, B. uniformis ATCC 8492 strain, M. elsdenit ATCC 25940 strain, R. gnavus ATCC 29149 strain, W. confusa ATCC 10881 strain, E. faecalis ATCC 29212 strain, A. muciniphila ATCC BAA-835 strain, and Prevotella copri ATCC BAA-1639 strain are purchased from Guangdong Microbial Culture Collection Center and Ningbo Mingzhou Biotechnology Co., Ltd., respectively.

EA is purchased from Sigma-Aldrich, Inc.

### EXAMPLE 1

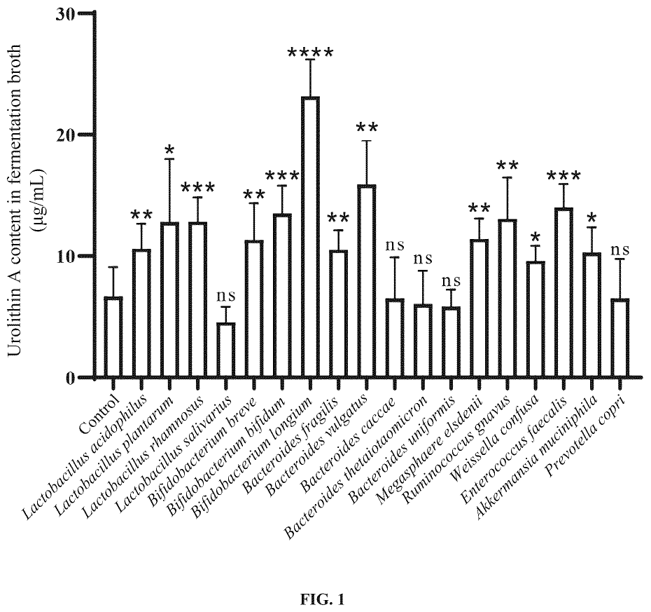

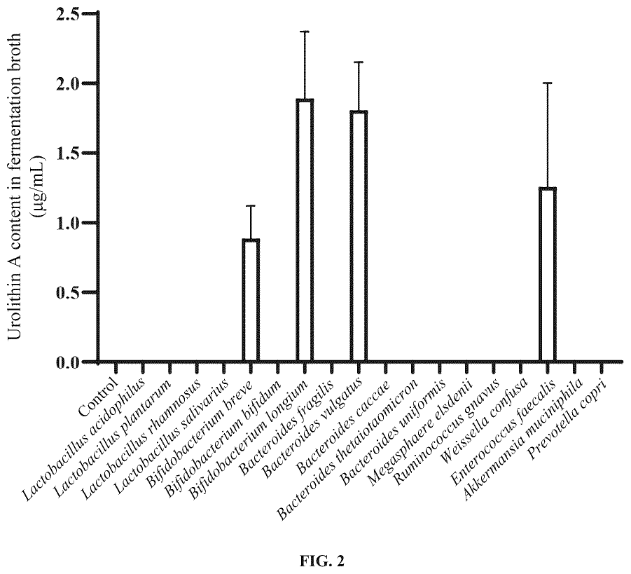

Feces (1 g) of metabotypes B and 0 populations were dissolved in 10 mL of phosphate buffered saline (PBS) to prepare 10% fecal suspensions, respectively; 2 mL of each suspension was added to 18 mL of BHI Broth supplemented with 1% EA, followed by adding 200 μL each of BHI-activated L. acidophilus, L. plantarum, L. rhamnosus, L. salivarius, B. breve, B. bifidum, B. longum, B. fragilis, B. vulgatus, B. caccae, B. thetaiotaomicron, B. uniformis, M. elsdenii, R. gnavus, W. confusa, E. faecalis, A. muciniphila, and Prevotella copri; the resulting mixed suspension was fermented in a 50 mL centrifuge tube at 37° C. for 48 h; 2 mL of fermentation broth was collected at 0, 24, and 48 h, respectively; the fermentation broth was centrifuged at 8,000 rpm for 10 min at 4° C.; the supernatant was added with dimethyl sulfoxide (DMSO) in a ratio of 1:1 and filtered through a 0.22 μm organic membrane, and the yield of urolithin A was assayed by high performance liquid chromatography (HPLC). It was found that L. acidophilus, L. plantarum, L. rhamnosus, B. breve, B. bifidum, B. longum, B. fragilis, B. vulgatus, M. elsdenii, R. gnavus, W. confusa, E. faecalis, and A. muciniphila could effectively increase the yield of urolithin A in the metabotype B population. Of them, B. breve, B. longum, B. vulgatus, and E. faecalis could regulate the metabolic production of urolithin A in the metabotype 0 population. The above 13 probiotics capable of improving the metabolism in the metabotype B population were randomly combined to investigate the in vitro regulation of microflora in metabotypes B and 0 populations. The results in metabotype B population is shown in FIG. 1, and the results in metabotype 0 population is shown in FIG. 2.

### EXAMPLE 2

A method for increasing urolithin A by a combination of L. acidophilus, L. plantarum, and L. rhamnosus with EA was provided, including the following steps:

feces (1 g) of metabotypes B and 0 populations were dissolved in 10 mL of PBS to prepare 10% fecal suspensions, respectively; 2 mL of each suspension was added to 18 mL of BHI Broth supplemented with 1% EA, followed by adding 200 μL of a mixed suspension of BHI-activated L. acidophilus, L. plantarum, and L. rhamnosus (the three strains were added in a ratio of 1:1:1, and the control group was added with an equivalent volume of PBS); the resulting mixed suspension was fermented in a 50 mL centrifuge tube at 37° C. for 48 h; 2 mL of fermentation broth was collected at 0, 24, and 48 h, respectively; the fermentation broth was centrifuged at 8,000 rpm for 10 min at 4° C.; the supernatant was added with DMSO in a ratio of 1:1 and filtered through a 0.22 μm organic membrane, and the yield of urolithin A was assayed by HPLC. The HPLC of urolithin A was performed as follows:

The liquid chromatograph was Waters e2695 HPLC System; the detector was Waters 2489 UV/Vis Detector; the detection wavelength was 305 nm; the chromatographic column was Eclipse XDB-C18 Column (250×4.6 mm. 5.0 μm; Agilent); the column temperature was 30° C.; the sample size was 10 μL; and the elution speed was 1.0 mL/min.

The mobile phases were 0.5% formic acid in water (A) and acetonitrile (B). Namely, A was composed of 0.5% formic acid and 99.5% double distilled water, while B was composed of 100% acetonitrile, where % represents volume percentage.

The elution gradient was programmed as follows: 0-20 min, 5%-25% B; 20-25 min, 25%-70% B; 25-26 min, 70%-5% B; and 26-35 min, 5% B.

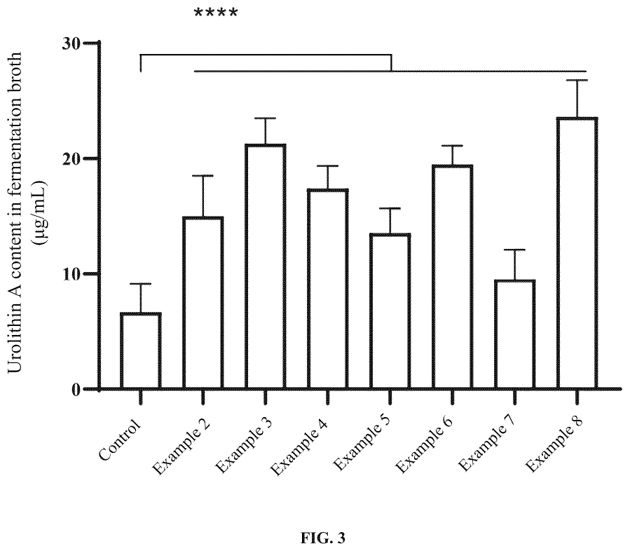

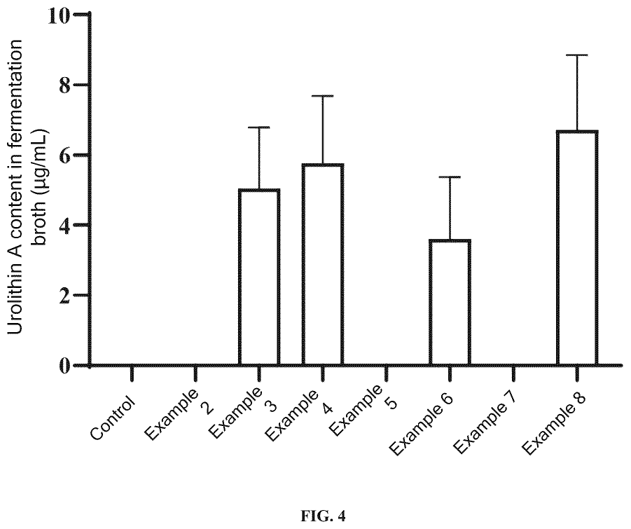

The results of Example 2 are shown in FIGS. 3 and 4. Compared with the control group, it was indicated that the content of urolithin A produced by metabolism in the metabotype B microflora increased from 6.69 μg/mL to 15.02 μg/mL. The metabotype 0 microflora was still unable to produce urolithin A after probiotics intervention.

### EXAMPLE 3

A method for increasing urolithin A by a combination of B. breve, B. bifidum, and B. longum with EA was provided, including the following steps:

feces (1 g) of metabotypes B and 0 populations were dissolved in 10 mL of PBS to prepare 10% fecal suspensions, respectively; 2 mL of each suspension was added to 18 mL of BHI Broth supplemented with 1% EA, followed by adding 200 μL of a mixed suspension of BHI-activated B. breve, B. bifidum, and B. longum (the three strains were added in a ratio of 1:1:1, and the control group was added with an equivalent volume of PBS); the resulting mixed suspension was fermented in a 50 mL centrifuge tube at 37° C. for 48 h; 2 mL of fermentation broth was collected at 0, 24, and 48 h, respectively; the fermentation broth was centrifuged at 8,000 rpm for 10 min at 4° C.; the supernatant was added with DMSO in a ratio of 1:1 and filtered through a 0.22 μm organic membrane, and the yield of urolithin A was assayed by HPLC. The HPLC assay was the same as that in Example 1.

The results of Example 3 are shown in FIGS. 3 and 4. Compared with the control group, the content of urolithin A produced by metabolism in the metabotype B microflora increased from 6.69 μg/mL to 21.26 μg/mL. The metabotype 0 microflora began to produce urolithin A (5.05 μg/mL) after probiotics intervention.

### EXAMPLE 4

A method for increasing urolithin A by a combination of B. fragilis and B. vulgatus with EA was provided, including the following steps:
feces (1 g) of metabotypes B and 0 populations were dissolved in 10 mL of PBS to prepare 10% fecal suspensions, respectively; 2 mL of each suspension was added to 18 mL of BHI Broth supplemented with 1% EA, followed by adding 200 μL of a mixed suspension of BHI-activated B. fragilis and B. vulgatus (the two strains were added in a ratio of 1:1, and the control group was added with an equivalent volume of PBS); the resulting mixed suspension was fermented in a 50 mL centrifuge tube at 37° C. for 48 h; 2 mL of fermentation broth was collected at 0, 24, and 48 h, respectively; the fermentation broth was centrifuged at 8,000 rpm for 10 min at 4° C.; the supernatant was added with DMSO in a ratio of 1:1 and filtered through a 0.22 μm organic membrane, and the yield of urolithin A was assayed by HPLC. The HPLC assay was the same as that in Example 1.

The results of Example 4 are shown in FIGS. 3 and 4. Compared with the control group, the content of urolithin A produced by metabolism in the metabotype B microflora increased from 6.69 μg/mL to 17.39 μg/mL. The metabotype 0 microflora began to produce urolithin A (5.76 μg/mL) after probiotics intervention.

### EXAMPLE 5

A method for increasing urolithin A by a combination of M. elsdenit, R. gnavus, and W. confusa with EA was provided, including the following steps:

feces (1 g) of metabotypes B and 0 populations were dissolved in 10 mL of PBS to prepare 10% fecal suspensions, respectively; 2 mL of each suspension was added to 18 mL of BHI Broth supplemented with 1% EA, followed by adding 200 μL of a mixed suspension of BHI-activated M. elsdenii, R. gnavus, and W. confusa (the three strains were added in a ratio of 1:1:1, and the control group was added with an equivalent volume of PBS); the resulting mixed suspension was fermented in a 50 mL centrifuge tube at 37° C. for 48 h; 2 mL of fermentation broth was collected at 0, 24, and 48 h, respectively; the fermentation broth was centrifuged at 8,000 rpm for 10 min at 4° C.; the supernatant was added with DMSO in a ratio of 1:1 and filtered through a 0.22 μm organic membrane, and the yield of urolithin A was assayed by HPLC. The HPLC assay was the same as that in Example 1.

The results of Example 5 are shown in FIGS. 3 and 4. Compared with the control group, the content of urolithin A produced by metabolism in the metabotype B microflora increased from 6.69 μg/mL to 13.54 μg/mL. The metabotype 0 microflora was still unable to produce urolithin A after probiotics intervention.

### EXAMPLE 6

A method for increasing urolithin A by a combination of E. faecalis and A. muciniphila with EA was provided, including the following steps:
feces (1 g) of metabotypes B and 0 populations were dissolved in 10 mL of PBS to prepare 10% fecal suspensions, respectively; 2 mL of each suspension was added to 18 mL of BHI Broth supplemented with 1% EA, followed by adding 200 μL of a mixed suspension of BHI-activated E. faecalis and A. muciniphila (the two strains were added in a ratio of 1:1, and the control group was added with an equivalent volume of PBS); the resulting mixed suspension was fermented in a 50 mL centrifuge tube at 37° C. for 48 h; 2 mL of fermentation broth was collected at 0, 24, and 48 h, respectively; the fermentation broth was centrifuged at 8,000 rpm for 10 min at 4° C.; the supernatant was added with DMSO in a ratio of 1:1 and filtered through a 0.22 μm organic membrane, and the yield of urolithin A was assayed by HPLC. The HPLC assay was the same as that in Example 1.

The results of Example 6 are shown in FIGS. 3 and 4. Compared with the control group, the content of urolithin A produced by metabolism in the metabotype B microflora increased from 6.69 μg/mL to 19.45 μg/mL. The metabotype 0 microflora began to produce urolithin A (3.60 μg/mL) after probiotics intervention.

### EXAMPLE 7

A method for increasing urolithin A by a combination of B. breve, B. fragilis, and A. muciniphila with EA was provided, including the following steps:
feces (1 g) of metabotypes B and 0 populations were dissolved in 10 mL of PBS to prepare 10% fecal suspensions, respectively; 2 mL of each suspension was added to 18 mL of BHI Broth supplemented with 1% EA, followed by adding 200 μL of a mixed suspension of BHI-activated B. breve, B. fragilis, and A. muciniphila (the three strains were added in a ratio of 1:1:1, and the control group was added with an equivalent volume of PBS); the resulting mixed suspension was fermented in a 50 mL centrifuge tube at 37° C. for 48 h; 2 mL of fermentation broth was collected at 0, 24, and 48 h, respectively; the fermentation broth was centrifuged at 8,000 rpm for 10 min at 4° C.; the supernatant was added with DMSO in a ratio of 1:1 and filtered through a 0.22 μm organic membrane, and the yield of urolithin A was assayed by HPLC. The HPLC assay was the same as that in Example 1.

The results of Example 7 are shown in FIGS. 3 and 4. Compared with the control group, the content of urolithin A produced by metabolism in the metabotype B microflora increased from 6.69 μg/mL to 9.53 μg/mL. The metabotype 0 microflora was still unable to produce urolithin A after probiotics intervention.

### EXAMPLE 8

A method for increasing urolithin A by a combination of B. longum, R. gnavus, and B. vulgatus with EA was provided, including the following steps:
feces (1 g) of metabotypes B and 0 populations were dissolved in 10 mL of PBS to prepare 10% fecal suspensions, respectively; 2 mL of each suspension was added to 18 mL of BHI Broth supplemented with 1% EA, followed by adding 200 μL of a mixed suspension of BHI-activated B. longum, R. gnavus, and B. vulgatus (the three strains were added in a ratio of 1:1:1, and the control group was added with an equivalent volume of PBS); the resulting mixed suspension was fermented in a 50 mL centrifuge tube at 37° C. for 48 h; 2 mL of fermentation broth was collected at 0, 24, and 48 h, respectively; the fermentation broth was centrifuged at 8,000 rpm for 10 min at 4° C.; the supernatant was added with DMSO in a ratio of 1:1 and filtered through a 0.22 μm organic membrane, and the yield of urolithin A was assayed by HPLC. The HPLC assay was the same as that in Example 1.

The results of Example 8 are shown in FIGS. 3 and 4. Compared with the control group, the content of urolithin A produced by metabolism in the metabotype B microflora increased from 6.69 μg/mL to 23.63 μg/mL. The metabotype 0 microflora began to produce urolithin A (6.71 μg/mL) after probiotics intervention.

### EXAMPLE 9

Sixty-four specific-pathogen-free (SPF)-grade metabotype A C57BL/6 mice were selected. Ain93G purified diet was used as a basal diet; 0.06% EA was added, and the converted intake was 60 mg/Kg. All mice were acclimatized for one week and randomized into eight groups of eight mice. Mixed suspensions were prepared according to the proportion of mixed microfloras in Examples 2 to 8 and each administered to different groups of the mice by gavage at a dose of 1×10⁸ CFU/mouse/day. An equivalent volume of normal saline was administered to control mice by gavage for 14 days. All mice were given access to food and water ad libitum. Mouse feces were collected on the last day of the experiment, and EA metabolism in mice was monitored. The mouse feces were added to DMSO in a volume-mass ratio of 1:10, fully ground in a grinder, and centrifuged at 8,000 rpm for 10 min at 4° C.; the supernatant was filtered through a 0.22 μm organic membrane, followed by HPLC assay. The assay was the same as that in Example 1.

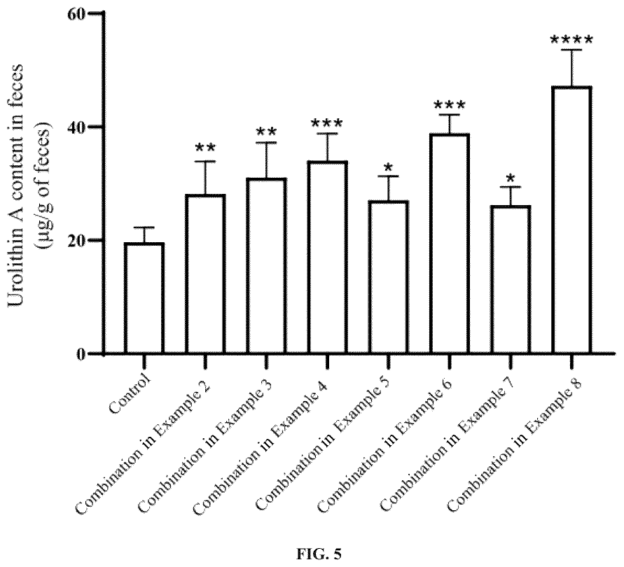

The results are shown in FIG. 5. Compared with the control group, all combinations could effectively increase the content of urolithin A produced by metabolism in mice.

The above descriptions are merely preferred embodiments of the present disclosure. It should be noted that a person of ordinary skill in the art may further make several improvements and modifications without departing from the principle of the present disclosure, but such improvements and modifications should be deemed as falling within the protection scope of the present disclosure.

(1-10). (canceled)

11. A pharmaceutical composition for increasing urolithin A content, comprising an intestinal probiotic and ellagic acid (EA), wherein the intestinal probiotic comprises at least one probiotic selected from the group consisting of Lactobacillus acidophilus, Lactobacillus plantarum, Lactobacillus rhamnosus, Bifidobacterium breve, Bifidobacterium bifidum, Bifidobacterium longium, Bacteroides fragilis, Bacteroides vulgatus, Megasphaere elsdenii, Ruminococcus gnavus, Weissella confusa, Enterococcus faecalis, and Akkermansia muciniphila.

12. The pharmaceutical composition according to claim 11, wherein the intestinal probiotic and EA have a volume-mass ratio of 1 mL:(5-20) g; and
the intestinal probiotic has a viable cell concentration of 1×10⁸ to 5×10⁸ clony-forming unit (CFU)/mL.

13. The pharmaceutical composition according to claim 11, wherein EA has a structure of formula I:

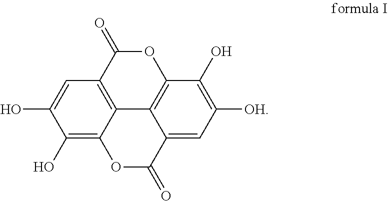 [SMILES: O=c1oc2c(O)c(O)cc3c(=O)oc4c(O)c(O)cc1c4c23]

14. The pharmaceutical composition according to claim 13, wherein the intestinal probiotic and the EA have a volume-mass ratio of 1 mL:(5-20) g; and
the intestinal probiotic has a viable cell concentration of 1×10⁸ to 5×10⁸ clony-forming unit (CFU)/mL.

15. The pharmaceutical composition according to claim 11, wherein the intestinal probiotic comprises the following combinations: a first intestinal probiotic formed by the Lactobacillus acidophilus, the Lactobacillus plantarum and the Lactobacillus rhamnosus, a second intestinal probiotic formed by the Bifidobacterium breve, the Bifidobacterium bifidum and the Bifidobacterium longium, a third intestinal probiotic formed by the Bacteroides fragilis and the Bacteroides vulgatus, a fourth intestinal probiotic formed by the Megasphaere elsdenii, the Ruminococcus gnavus and the Weissella confusa, a fifth intestinal probiotic formed by the Enterococcus faecalis and the Akkermansia muciniphila, a sixth intestinal probiotic formed by the Bifidobacterium breve, the Bacteroides fragilis and the Akkermansia muciniphila, and a seventh intestinal probiotic formed by the Bifidobacterium longium, the Ruminococcus gnavus and the Bacteroides vulgatus.

16. The pharmaceutical composition according to claim 11, wherein a ratio of viable counts of microbial species within the first intestinal probiotic, the second intestinal probiotic, the six intestinal probiotic, the seventh intestinal probiotic or the fourth intestinal probiotic is 1-3:1-3:1-3; and
a ratio of viable counts of microbial species within the third intestinal probiotic or the fifth intestinal probiotic is 1-3:1-3.

17. The pharmaceutical composition according to claim 11, wherein the intestinal probiotic promotes metabolism of EA into the urolithin A.

18. The pharmaceutical composition according to claim 11, wherein a population metabotype that promotes metabolism of EA into urolithin A is metabotype O and/or B;
at least one selected from the group consisting of the first intestinal probiotic to the seventh intestinal probiotic is prepared into a medicament that promotes metabolism of EA into urolithin A in a metabotype B population; and
at least one selected from the group consisting of the second intestinal probiotic, the third intestinal probiotic, the fifth intestinal probiotic and the seventh intestinal probiotic is prepared into a medicament that promotes metabolism of EA into urolithin A in a metabotype O population.

19. A method for preventing and/or treating a disease selected from the group consisting of diabetes, obesity, senescence, central nervous system lesions and viral infection, comprising administering to a patient in need thereof a therapeutically effective dose of the pharmaceutical composition according to claim 11.

20. The method according to claim 19, wherein the patient belongs to a metabotype B population and/or a metabotype O population of EA metabolism.

21. The method according to claim 19, wherein the intestinal probiotic comprises at least one probiotic selected from the group consisting of Lactobacillus acidophilus, Lactobacillus plantarum, Lactobacillus rhamnosus, Bifidobacterium breve, Bifidobacterium bifidum, Bifidobacterium longium, Bacteroides fragilis, Bacteroides vulgatus, Megasphaere elsdenii, Ruminococcus gnavus, Weissella confusa, Enterococcus faecalis, and Akkermansia muciniphila.

22. The method according to claim 19, wherein the intestinal probiotic and the EA have a volume-mass ratio of 1 mL:(5-20) g; and
the intestinal probiotic has a viable cell concentration of 1×10⁸ to 5×10⁸ clony-forming unit (CFU)/mL.

23. The method according to claim 19, wherein EA has a structure of formula I:

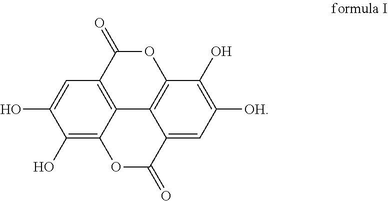 [SMILES: O=c1oc2c(O)c(O)cc3c(=O)oc4c(O)c(O)cc1c4c23]

24. The method according to claim 22, wherein the intestinal probiotic and the EA have a volume-mass ratio of 1 mL:(5-20) g; and
the intestinal probiotic has a viable cell concentration of 1×10⁸ to 5×10⁸ clony-forming unit (CFU)/mL.

25. The method according to claim 19, wherein the intestinal probiotic comprises the following combinations: a first intestinal probiotic formed by the Lactobacillus acidophilus, the Lactobacillus plantarum and the Lactobacillus rhamnosus, a second intestinal probiotic formed by the Bifidobacterium breve, the Bifidobacterium bifidum and the Bifidobacterium longium, a third intestinal probiotic formed by the Bacteroides fragilis and the Bacteroides vulgatus, a fourth intestinal probiotic formed by the Megasphaere elsdenii, the Ruminococcus gnavus and the Weissella confusa, a fifth intestinal probiotic formed by the Enterococcus faecalis and the Akkermansia muciniphila, a sixth intestinal probiotic formed by the Bifidobacterium breve, the Bacteroides fragilis and the Akkermansia muciniphila, and a seventh intestinal probiotic formed by the Bifidobacterium longium, the Ruminococcus gnavus and the Bacteroides vulgatus.

26. The method according to claim 19, wherein a ratio of viable counts of microbial species within the first intestinal probiotic, the second intestinal probiotic, the six intestinal probiotic, the seventh intestinal probiotic or the fourth intestinal probiotic is 1-3:1-3:1-3; and
a ratio of viable counts of microbial species within the third intestinal probiotic or the fifth intestinal probiotic is 1-3:1-3.

27. The method according to claim 19, wherein the intestinal probiotic promotes metabolism of EA into the urolithin A.

28. The method according to claim 19, wherein a population metabotype that promotes metabolism of EA into urolithin A is metabotype O and/or B;
at least one selected from the group consisting of the first intestinal probiotic to the seventh intestinal probiotic is prepared into a medicament that promotes metabolism of EA into urolithin A in a metabotype B population; and
at least one selected from the group consisting of the second intestinal probiotic, the third intestinal probiotic, the fifth intestinal probiotic and the seventh intestinal probiotic is prepared into a medicament that promotes metabolism of EA into urolithin A in a metabotype O population.
